SEQUENCE LISTING (2 sequences)

SEQ ID NO: 1 - DNA, 19 bp, synthetic construct
  source 1..19: mol_type = other DNA; note = forward primer for detecting EHP
gcagcactcaaggaatggc

SEQ ID NO: 2 - DNA, 22 bp, synthetic construct
  source 1..22: mol_type = other DNA; note = reverse primer for detecting EHP
tttcgttaggcttaccctgtga

(19) United States
(12) Patent Application Publication
(10) Pub. No.: US 2026/0069645 A1
(43) Pub. Date: Mar. 12, 2026

(54) METHOD AGAINST ENTEROCYTOZOON HEPATOPENAEI INFECTION

(71) Applicant: SYNBIO TECH INC., Kaohsiung City (TW)

(72) Inventors: Jin-Seng LIN, Tainan City (TW); Hsiao-Tung CHANG, Tainan City (TW)

(21) Appl. No.: 19/288,065

(22) Filed: Aug. 1, 2025

Related U.S. Application Data
(63) Continuation of application No. PCT/CN2025/099894, filed on Jun. 9, 2025.
(60) Provisional application No. 63/693,454, filed on Sep. 11, 2024.

(51) Int. Cl. A61K 35/747; A61P 31/10

(52) U.S. Cl. CPC A61K 35/747; A61P 31/10

(57) ABSTRACT

A method against Enterocytozoon hepatopenaei infection includes administering to a subject in need thereof a composition containing Enterococcus faecium EF08 which is deposited at the China General Microbiological Culture Collection Center (CGMCC) under an accession number CGMCC 5549, Lactobacillus acidophilus LA1063 which is deposited at the CGMCC under an accession number CGMCC 18744, and Lactiplantibacillus plantarum LP28 which is deposited at the CGMCC under an accession number CGMCC 3346 in accordance with the Budapest Treaty.

### CROSS-REFERENCE TO RELATED APPLICATION

This application is a continuation application of International Application No. PCT/CN2025/099894, filed on Jun. 9, 2025, which claims priority to U.S. Provisional Patent Application No. 63/693,454, filed on Sep. 11, 2024. The aforesaid applications are incorporated by reference herein in their entirety.

### SEQUENCE LISTING XML

The Sequence Listing submitted concurrently herewith with a file name of “PE-71867-AM-SEQUENCE LISTING.xml,” a creation date of Jun. 13, 2025, and a size of 3.16 kilobytes, is part of the specification and is incorporated by reference in its entirety.

### FIELD

The disclosure relates to a method against Enterocytozoon hepatopenaei infection using a composition containing Enterococcus faecium EF08, Lactobacillus acidophilus LA1063, and Lactiplantibacillus plantarum LP28.

### BACKGROUND

Enterocytozoon hepatopenaei (EHP) is a fungal species from genus Enterocytozoon in the family Enterocytozoonidae. Hosts infected with EHP are mainly aquaculture animals such as shrimps and crabs, and EHP usually parasitizes in hepatopancreas of such hosts. EHP Infection may occur through ingestion of EHP-contaminated water, food, or feces. Hosts infected with EHP usually suffer from reduced food intake, growth retardation, and damage to the hepatopancreas, which are accompanied by symptoms such as white feces and white turbidity in muscles, and in severe cases, death may even occur.

Currently, there is no effective way to fight against EHP infection, and preventive strategies, for example, paying attention to the management regarding feeding and water quality, strengthening the disinfection of breeding ponds, etc., are usually used to reduce the occurrence of EHP infection. However, the effects of such preventive strategies in reducing the occurrence of EHP infection are still not satisfactory.

Probiotics, which may serve as a live microbial food ingredient, are beneficial to health, and can selectively stimulate the growth of native bacteria in an intestine of a subject, so as to change the composition, quantity or nature of intestinal microflora of the subject, thereby enhancing intestinal health and immune response thereof. At present, microorganisms known to be used as probiotics may include Lactobacillus, Enterococcus, Bifidobacterium, Bacillus, Lactococcus, Enterococcus, Saccharomyces, Streptococcus, and so forth.

In spite of the aforesaid, there is still a need to develop a method that is effective against EHP infection.

### SUMMARY

Accordingly, the present disclosure provides a method against Enterocytozoon hepatopenaei infection, which can alleviate at least one of the drawbacks of the prior art, and which includes administering to a subject in need thereof a composition containing Enterococcus faecium EF08 which is deposited at the China General Microbiological Culture Collection Center (CGMCC) under an accession number CGMCC 5549, Lactobacillus acidophilus LA1063 which is deposited at the CGMCC under an accession number CGMCC 18744, and Lactiplantibacillus plantarum LP28 which is deposited at the CGMCC under an accession number CGMCC 3346 in accordance with the Budapest Treaty.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

In the development of approaches to fight against Enterocytozoon hepatopenaei (EHP) infection, the applicant surprisingly found that a composition containing Enterococcus faecium EF08 which is deposited at the China General Microbiological Culture Collection Center (CGMCC) under an accession number CGMCC 5549, Lactobacillus acidophilus LA1063 which is deposited at the CGMCC under an accession number CGMCC 18744, and Lactiplantibacillus plantarum LP28 (also known as Lactobacillus plantarum LP28) which is deposited at the CGMCC under an accession number CGMCC 3346 can effectively inhibit the growth of EHP in a white shrimp (i.e., Penaeus vannamei), and hence is expected to be effective against EHP infection.

Therefore, the present disclosure provides a method against Enterocytozoon hepatopenaei (EHP) infection, which includes administering to a subject in need thereof a composition containing Enterococcus faecium EF08 which is deposited at the China General Microbiological Culture Collection Center (CGMCC) under an accession number CGMCC 5549, Lactobacillus acidophilus LA1063 which is deposited at the CGMCC under an accession number CGMCC 18744, and Lactiplantibacillus plantarum LP28 which is deposited at the CGMCC under an accession number CGMCC 3346 in accordance with the Budapest Treaty.

As used herein, the terms “against Enterocytozoon hepatopenaei infection” and “anti-Enterocytozoon hepatopenaei infection” can be interchangeably used, and are intended to cover the prevention of infection caused by Enterocytozoon hepatopenaei, the inhibition of replication of Enterocytozoon hepatopenaei, and the treatment and/or prevention of infectious diseases caused by Enterocytozoon hepatopenaei.

As used herein, the term “administering” or “administration” means introducing, providing or delivering the abovementioned composition to a subject by any suitable routes to perform its intended function.

As used herein, the term “subject” refers to an aquaculture animal, e.g., a shrimp.

In certain embodiments, the shrimp may be selected from the group consisting of Penaeus vannamei, Penaeus monodon, Macrobrachium rosenbergii, Litopenaeus stylirostris, and combinations thereof. In an exemplary embodiment, the shrimp is Penaeus vannamei.

According to the present disclosure, Enterococcus faecium EF08, Lactobacillus acidophilus LA1063, and Lactiplantibacillus plantarum LP28 may be live cells or dead cells, concentrated or non-concentrated, a liquid, a paste, a semi-solid, or a solid (e.g., a pellet, a granule, or a powder), and may be heat-inactivated, frozen, dried, or freeze-dried (e.g., may be in freeze-dried form or spray/fluid bed dried form). In an exemplary embodiment, each of Enterococcus faecium EF08, Lactobacillus acidophilus LA1063, and Lactiplantibacillus plantarum LP28 is in freeze-dried form.

In certain embodiments, a ratio of a number of Enterococcus faecium EF08, to a number of Lactobacillus acidophilus LA1063, and to a number of Lactiplantibacillus plantarum LP28 may range from 1:0.01:1 to 1:0.03:3. In an exemplary embodiment, the ratio of the number of Enterococcus faecium EF08, to that of Lactobacillus acidophilus LA1063, and to that of Lactiplantibacillus plantarum LP28 is 1:0.01:3.

According to the present disclosure, the composition may have a total bacterial concentration ranging from 1×10⁸ CFU/g to 1×10¹¹ CFU/g. In certain embodiments, the composition may have a total bacterial concentration ranging from 1×10⁸ CFU/g to 1×10¹⁰ CFU/g. In an exemplary embodiment, the composition has a total bacterial concentration of 1×10⁸ CFU/g.

According to the present disclosure, the composition may be prepared in the form of a pharmaceutical composition.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, wetting agents, absorption delaying agents, liposomes, and the like. The choice and amount of the aforesaid agents are within the expertise and routine skills of those skilled in the art. In an exemplary embodiment, the pharmaceutical composition further includes a maltodextrin serving as an excipient.

According to the present disclosure, the composition may be directly administered to a water body where the aquaculture animal is raised, or may be added to a feed of the aquaculture animal using techniques well known to those skilled in the art, and then administered to the water body. For example, the composition may be directly added to the feed of the aquaculture animal, or may be used to prepare an intermediate composition (e.g., a feed additive or a premix) suitable to be subsequently added to the feed. In an exemplary embodiment, the composition is administered to the water body after being mixed with the feed of the aquaculture animal.

According to the present disclosure, the composition may be added to the feed of the aquaculture animal in an amount ranging from 0.001% (w/w) to 1% (w/w), followed by mixing. In an exemplary embodiment, the composition is added to the feed for the aquaculture animal in an amount of 0.1% (w/w), followed by mixing.

According to the present disclosure, the feed may be self-prepared, or may be a commercially available product. Examples of the feed may include, but are not limited to, fish meal, soybean meal, peanut meal, chicken meal, blood meal, squid meal, α-starch, DL-methionine (99%), lysine (99%), a vitamin premix, and a mineral mixture.

The present disclosure also provides the aforesaid composition for use against Enterocytozoon hepatopenaei infection. The use includes administering to a subject in need thereof the aforesaid composition.

The present disclosure further provides use of the aforesaid composition in the manufacture of a medication or an animal feed against Enterocytozoon hepatopenaei infection in a subject.

As used herein, the term “animal feed” refers to any kind of material, liquid or solid, which is used for nourishing an animal, and for sustaining normal or accelerated growth of an animal including newborns and young developing animals. In certain embodiments, the animal feed is an aquaculture feed.

The disclosure will be further described by way of the following examples. However, it should be understood that the following examples are intended solely for the purpose of illustration and should not be construed as limiting the present disclosure in practice.

### EXAMPLES

### General Experimental Materials:

### 1. Probiotic Strains

The probiotic strains used in the following examples, i.e., Enterococcus faecium EF08, Lactobacillus acidophilus LA1063, and Lactiplantibacillus plantarum LP28 (also known as Lactobacillus plantarum LP28), have been deposited at the Bioresource Collection and Research Center (BCRC) of the Food Industry Research and Development Institute (FIRDI) (No. 331, Shih-Pin Rd., Hsinchu City 300, Taiwan), and have also been deposited at the China General Microbiological Culture Collection Center (CGMCC) of Chinese Academy of Sciences, the Institute of Microbiology (No. 1, West Beichen Rd., Chaoyang District, Beijing 100101, China) in accordance with the Budapest Treaty.

The relevant information regarding each of the probiotic strains (including accession number and date of deposit) is listed in Table 1 below.

TABLE 1

| Probiotic strains | Accession number | Date of deposit |
|---|---|---|
| Enterococcus | BCRC 910525 | Sep. 2, 2011 |
| faecium EF08 | CGMCC 5549 | Dec. 8, 2011 |
| Lactobacillus | BCRC 911041 | Mar. 9, 2021 |
| acidophilus LA1063 | CGMCC 18744 | Oct. 28, 2019 |
| Lactiplantibacillus | BCRC 910435 | Jul. 14, 2009 |
| plantarum LP28* | CGMCC 3346 | Oct. 19, 2009 |

*Lactiplantibacillus plantarum LP28 has been disclosed in TW I662902 B and CN 108967269 B, and is readily available to the public

### Example 1. Preparation of Composition Containing Enterococcus faecium EF08, Lactobacillus acidophilus LA1063, and Lactiplantibacillus plantarum LP28 According to the Present Disclosure

First, each of Enterococcus faecium EF08, Lactobacillus acidophilus LA1063, and Lactiplantibacillus plantarum LP28 described in Section 1 of General Experimental Materials was inoculated at an amount of 1% (v/v) into BD Difco™ Lactobacilli MRS broth (Cat. No. 288130), and was then cultivated under an anaerobic condition at 37° C. for 24 hours, so as to obtain a liquid culture. After centrifugation at 3000 rpm and 25° C. for 10 minutes, the cell pellet was collected, and was washed with an appropriate amount of a physiological saline solution. After that, the cell pellet was suspended in 9 mL of a saline solution to obtain a suspension, followed by performing cell counting using a plate counting medium. Next, the suspension was subjected to centrifugation at 3000 rpm and 25° C. for 10 minutes, followed by removing a supernatant, so as to obtain the resultant cell pellet. After that, the resultant cell pellet was subjected to lyophilization, thereby obtaining a powder of Enterococcus faecium EF08 (hereinafter abbreviated as “Enterococcus faecium EF08 powder”), a powder of Lactobacillus acidophilus LA1063 (hereinafter abbreviated as “Lactobacillus acidophilus LA1063 powder”), and a powder of Lactiplantibacillus plantarum LP28 (hereinafter abbreviated as “Lactiplantibacillus plantarum LP28 powder”), each of which had a bacterial concentration of 1×10¹¹ CFU/g.

Thereafter, the Enterococcus faecium EF08 powder, the Lactobacillus acidophilus LA1063 powder, and the Lactiplantibacillus plantarum LP28 powder were mixed at a number ratio of 1:0.01:3 to obtain a bacterial mixture, and then the bacterial mixture was mixed with a maltodextrin, which served as a carrier of the bacterial mixture, at a weight ratio of 1:999, thereby obtaining a composition that contains Enterococcus faecium EF08, Lactobacillus acidophilus LA1063, and Lactiplantibacillus plantarum LP28, and that has a bacterial concentration of 1×10⁸ CFU/g.

### Example 2. Evaluation of the Efficacy of the Composition According to the Present Disclosure Against Enterocytozoon hepatopenaei (EHP) Infection

### Experimental Materials:

### 1. Experimental Shrimps

The experimental shrimps (i.e., Penaeus vannamei) (56 days old, weighting approximately 1.4 g) used in this example were obtained from the shrimp farm of the National Pingtung University of Science and Technology. All the experimental shrimps were housed in a water tank (volume: 60 L) containing 40 L of seawater with a salinity of 25 psu under the following conditions: an alternating 12-hour light and 12-hour dark cycle, a seawater temperature maintained at 27±1° C., and continuous oxygen supply. Furthermore, feed was provided ad libitum for all the experimental shrimps.

### 2. Preparation of EHP-Infected Hepatopancreas Tissues

First, hepatopancreases were removed from EHP-infected Penaeus vannamei and cut into pieces, thereby obtaining EHP-infected hepatopancreas tissues (EHP load: 2.85±1.77×10⁵ copy number/ng DNA).

### Experimental Procedures:

### A. Administration of the Composition According to the Present Disclosure

The experimental shrimps (i.e., Penaeus vannamei) were randomly divided into 2 groups (n=10 in each group), including a control group and an experimental group. A standard diet containing ingredients shown in Table 2 below was provided for preparing a probiotic feed and a control feed to be administered to the experimental shrimps in the experimental group and the control group, respectively (dose: 0.1 g/g body weight/day). Specifically, the composition containing Enterococcus faecium EF08, Lactobacillus acidophilus LA1063, and Lactiplantibacillus plantarum LP28 obtained in Example 1 was added to the standard diet at an amount of 0.1% (w/w), and was mixed evenly with the standard diet, thereby obtaining the probiotic feed to be administered to the experimental shrimps in the experimental group. Additionally, a maltodextrin was added to the standard diet at an amount of 0.1% (w/w), thereby obtaining the control feed to be administered to the experimental shrimps in the control group. The experimental shrimps in each group were fed with the probiotic feed or the control feed twice a day, and the administration was conducted for a period of 63 days.

TABLE 2

| Ingredient | Amount (g/kg) |
|---|---|
| Fish meal (crude protein: 68%) | 90 |
| Fish meal (crude protein: 64%) | 200 |
| Soybean meal | 230 |
| Peanut meal | 100 |
| Chicken meal | 90 |
| Blood meal | 10 |
| Squid meal | 56 |
| α-starch | 200 |
| DL-methionine (99%) | 2 |
| Lysine (99%) | 4 |
| Vitamin premix^{a} | 4 |
| Mineral mixture^{b} | 13 |

^{a}5.15 g of calcium pantothenate, 0.0037 g of cholecalciferol, 153.8 g of choline chloride, 0.0074 g of cyanocobalamin, 10 g of DL-α-tocopheryl acetate, 0.09 g of d-biotin, 0.08 g of folic acid, 100 g of inositol, 1.5 g of menadione, 0.27 g of nicotinic acid, 10 g of para-aminobenzoic acid, 1.5 g of pyridoxine hydrochloride, 0.64 g of retinol acetate, 0.83 g of riboflavin, and 0.52 g of thiamine hydrochloride were mixed in α-cellulose
^{b}200 g of K₂HPO₄, 215 g of NaH₂PO₄, 265 g of Ca(H₂PO₄)₂•H₂O, 105 g of CaCO₃, 28 g of KCl, 0.233 g of Kl, 100 g of MgSO₄•7H₂O, 0.153 g of CuCl₂•2H₂O, 0.24 g of AlCl₃•6H₂O, and 0.24 g of ZnSO₄•7H₂O were mixed in α-cellulose

B. Infection with EHP

On the 56^{th} after administration of the probiotic feed or the control feed, the experimental shrimps in each group were also fed with the EHP-infected hepatopancreas tissues obtained in Section 2 of Experimental Materials of this example (dosage: 0.1 g of the EHP-infected hepatopancreas tissue/i g body weight) twice the day, so that the experimental shrimps in each group were infected with EHP.

### C. Determination of EHP Load

After 63 days of administration of the probiotic feed or the control feed, five experimental shrimps were randomly selected from each group, followed by obtaining hepatopancreas tissues thereof. The hepatopancreas tissues thus obtained in each group were then subjected to extraction of genomic DNA using Gene-Spin™-V³ Genomic DNA isolation kit (Protech Technology Enterprise Co., Ltd.) according to the manufacturer's instructions. The resultant genomic DNA of each group, serving as a template, was subjected to quantitative real-time polymerase chain reaction (quantitative real-time PCR), which was performed on an ABI PRISM 7900 Sequence Detection System (Applied Biosystems) using a designed primer pair specific for EHP shown in Table 3 below, with reference to Wang L. et al. (2020), Microorganisms, 8:1366, doi: 10.3390/microorganisms8091366., and which was performed under the reaction conditions shown in Table 4 below.

TABLE 3

| PCR primer | Nucleotide sequence | Size of PCR |
|---|---|---|
| pair | (5′→3′) | product (bp) |
| Forward | gcagcactcaaggaatggc | 238 |
| primer | (SEQ ID NO: 1) | |
| Reverse | tttcgttaggcttaccctgtga | |
| primer | (SEQ ID NO: 2) | |

TABLE 4

| Reaction mix | Volume (μL) |
|---|---|
| DNA (100 ng/μL) | 2 |
| Forward primer (0.2 μM) | 2 |
| Reverse primer (0.2 μM) | 2 |
| 2x qPCRBIO SyGreen Blue Mix Hi-ROX | 10 |
| (PCR Biosystems) | |
| Diethyl pyrocarbonate (DEPC)-treated water | 4 |

Operation conditions: denaturation at 95° C. for 5 minutes, followed by 40 cycles of the following reactions: denaturation at 95° C. for 10 seconds, primer annealing at 60° C. for 30 seconds, and extension at 95° C. for 15 seconds

The cycle threshold (Ct) value thus determined in each group was then converted into an EHP load (copy number/ng DNA) of the hepatopancreas tissues based on a standard curve of DNA standards with different known DNA loads (ranging from 1.0×10⁸ to 1.0×10¹ copy number) plotted against Ct value of their own.

In this example, the experiments were performed in triplicates, and the experimental data thus obtained are expressed as mean±standard deviation (SD), and were analyzed using Tukey's test, so as to evaluate the differences between the groups. Statistical significance is indicated by p<0.05.

### Results:

Referring to Table 5 below, the EHP load of the hepatopancreas tissues determined in the experimental group was significantly reduced compared with that of the control group. These results demonstrate that the composition according to the present disclosure, which contains Enterococcus faecium EF08, Lactobacillus acidophilus LA1063, and Lactiplantibacillus plantarum LP28, can effectively inhibit the growth of EHP in bodies of Penaeus vannamei, and hence is effective against EHP infection.

TABLE 5

| Group | EHP load (copy number/ng DNA) |
|---|---|
| Control group | 6.58 ± 4.5 × 10³ |
| Experimental group | 3.16 ± 1.38 × 10²* |

*indicates that p < 0.05 when compared with the control group

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method against Enterocytozoon hepatopenaei infection, comprising administering to a subject in need thereof a composition containing Enterococcus faecium EF08 which is deposited at the China General Microbiological Culture Collection Center (CGMCC) under an accession number CGMCC 5549, Lactobacillus acidophilus LA1063 which is deposited at the CGMCC under an accession number CGMCC 18744, and Lactiplantibacillus plantarum LP28 which is deposited at the CGMCC under an accession number CGMCC 3346 in accordance with the Budapest Treaty.

2. The method as claimed in claim 1, wherein a ratio of a number of Enterococcus faecium EF08, to a number of Lactobacillus acidophilus LA1063, and to a number of Lactiplantibacillus plantarum LP28 is 1:0.01:3.
